(19) United States
(12) Patent Application Publication
(10) Pub. No.: US 2025/0107740 A1
(43) Pub. Date: Apr. 3, 2025

(54) METHOD AND SYSTEM FOR FUNCTIONAL AND STRUCTURAL REMODELING PROGRESSION IN ATRIAL FIBRILLATION

(71) Applicant: Northwestern University, Evanston, IL (US)

(72) Inventor: Markus Rottmann, Chicago, IL (US)

(21) Appl. No.: 18/901,448

(22) Filed: Sep. 30, 2024

Related U.S. Application Data
(60) Provisional application No. 63/541,443, filed on Sep. 29, 2023.

(51) Int. Cl. A61B 5/361; A61B 5/00; G16H 20/00

(52) U.S. Cl. CPC A61B 5/361; A61B 5/4035; A61B 5/7235; A61B 5/7275; A61B 5/7282; G16H 20/00

(57) ABSTRACT

A system to identify and predict atrial fibrillation includes a memory configured to store one or more electrograms and one or more nerve recordings of a patient. The system also includes a processor operatively coupled to the memory and configured to identify one or more atrial fibrillation characteristics based on the one or more electrograms and the one or more nerve recordings, where the one or more atrial fibrillation characteristics include oxidative stress and fibrosis. The processor is also configured to identify a progression state of the atrial fibrillation based on the one or more atrial fibrillation characteristics.

### CROSS-REFERENCE TO RELATED APPLICATION

The present application claims the priority benefit of U.S. Provisional Patent App. No. 63/541,443 filed on Sep. 29, 2023, the entire disclosure of which is incorporated by reference herein.

### BACKGROUND

Atrial fibrillation (AF) can be classified as paroxysmal, persistent, longstanding persistent, and permanent. Paroxysmal AF is defined as recurrent AF (2 episodes) that terminates spontaneously within 7 days. Persistent AF is defined as continuous AF that is sustained beyond 7 days. Continuous AF for longer than a year is described as longstanding persistent AF. Common conventional predictors of AF progression from sinus rhythm to paroxysmal and persistent as well as permanent AF include age 65-74 years, hypertension, transient ischemic attack or stroke, chronic obstructive pulmonary disease, heart failure, diabetes mellitus, vascular disease, sex category scores, as well as biomarkers related to inflammation. In an early AF remodeling stage, AF is determined through electrophysiological, structural, and mechanical remodeling by reducing the atrial effective refractory periods, by the depression of intra-atrial conduction and the loss of contractile function contributing to the perpetuation of AF, and through the progression from paroxysmal to persistent and permanent AF. It has been shown that the longer patients wait to implement a rhythm treatment strategy, the more difficult it is to regain sinus rhythm.

### SUMMARY

An illustrative system to identify and predict atrial fibrillation includes a memory configured to store one or more electrograms and one or more nerve recordings of a patient. The system also includes a processor operatively coupled to the memory and configured to identify one or more atrial fibrillation characteristics based on the one or more electrograms and the one or more nerve recordings, where the one or more atrial fibrillation characteristics include oxidative stress and fibrosis. The processor also identifies a progression state of the atrial fibrillation based on the one or more atrial fibrillation characteristics.

In one embodiment, the one or more atrial fibrillation characteristics include autonomic remodeling. In another embodiment, the memory is further configured to store one or more T1/T2 maps obtained through imaging, and the one or more atrial fibrillation characteristics are identified based at least in part on the one or more T1/T2 maps. In another embodiment, the processor predicts a subsequent progression state of the atrial fibrillation based on the identified progression state and the one or more atrial fibrillation characteristics.

In another embodiment, the memory stores an indication of whether the patient plans to pursue treatment of the atrial fibrillation, and the processor predicts a survival rate for the patient over a period of time, where the survival rate is based on the indication of whether the patient plans to pursue treatment and the identified progression state. In one embodiment, the processor is configured to identify an optimal treatment plan for the atrial fibrillation based on the identified progression state and the one or more atrial fibrillation characteristics. In another embodiment, the processor predicts a time at which the atrial fibrillation will terminate and a normal sinus rhythm will commence for the patient, where the prediction is based on the identified progression state. In another embodiment, the processor also predicts a duration of the normal sinus rhythm.

In another embodiment, the one or more atrial fibrillation characteristics include cycle length, organization index, dominant frequency, and voltage. In one embodiment, the processor uses telemetry to obtain and analyze the one or more nerve recordings. the one or more atrial fibrillation characteristics include oxidative stress levels in right and left atrial sub-regions of the patient, and wherein the progression state is identified based at least in part on the oxidate stress levels. In another embodiment, the one or more atrial fibrillation characteristics include a fat percentage in heart tissue of the patient, and the progression state is identified based at least in part on the fat percentage. In another embodiment, the one or more atrial fibrillation characteristics incudes nerve frequency, and the processor quantifies nerve frequency based at least in part on a time between nerve peaks assessed with zero-crossings based on the one or more nerve recordings. In another embodiment, the nerve frequency is based at least in part on an area under a nerve signal. In another embodiment, the one or more electrograms include an intracardiac electrogram of the patient and a body surface electrogram of the patient.

An illustrative method for identifying and predicting atrial fibrillation includes storing, in a memory of a computing system, one or more electrograms and one or more nerve recordings of a patient. The method also includes identifying, by a processor of the computing system, one or more atrial fibrillation characteristics based on the one or more electrograms and the one or more nerve recordings, where the one or more atrial fibrillation characteristics include oxidative stress and fibrosis. The method further includes identifying, by the processor, a progression state of the atrial fibrillation based on the one or more atrial fibrillation characteristics.

In another embodiment, the method includes storing, in the memory, one or more T1/T2 maps obtained through imaging, where the one or more atrial fibrillation characteristics are identified based at least in part on the one or more T1/T2 maps. In another embodiment, the method includes predicting, by the processor, a subsequent progression state of the atrial fibrillation based on the identified progression state and the one or more atrial fibrillation characteristics. In another embodiment, the method includes storing, in the memory, an indication of whether the patient plans to pursue treatment of the atrial fibrillation and predicting, by the processor, a survival rate for the patient over a period of time, where the survival rate is based on the indication of whether the patient plans to pursue treatment and the identified progression state. In another embodiment, the method incudes identifying, by the processor, an optimal treatment plan for the atrial fibrillation based on the identified progression state and the one or more atrial fibrillation characteristics.

### BRIEF DESCRIPTION OF THE DRAWINGS

Illustrative embodiments of the invention will hereafter be described with reference to the accompanying drawings, wherein like numerals denote like elements.

FIG. 1A depicts a high resolution mapping plaque in accordance with an illustrative embodiment.

FIG. 1B depicts an electrogram measure of cycle length (dependent on pacing days in the posterior left atrium) in accordance with an illustrative embodiment.

FIG. 1C depicts an electrogram measure of dominant frequency (dependent on pacing days in the posterior left atrium) in accordance with an illustrative embodiment.

FIG. 1D depicts an electrogram measure of organization index (dependent on pacing days in the posterior left atrium) in accordance with an illustrative embodiment.

FIG. 2A shows CL changes in the development of persistent AF dependent on pacing days in accordance with an illustrative embodiment.

FIG. 2B depicts that bipolar voltage (V_{bip}) decreased strongly over the number of RAP days (R=0.7, P<0.05) in the PLA not only pre- but also post-80 RAP days in accordance with an illustrative embodiment.

FIG. 2C depicts example V_{bip} maps at 50 RAP days in accordance with an illustrative embodiment.

FIG. 2D depicts example V_{bip} maps at 230 RAP days in accordance with an illustrative embodiment.

FIG. 2E is a histogram corresponding to FIG. 2C that shows the decrease in V_{bip} over RAP days in accordance with an illustrative embodiment.

FIG. 2F is a histogram corresponding to FIG. 2D that shows the decrease in V_{bip} over RAP days in accordance with an illustrative embodiment.

FIG. 3A shows a schematic of parasympathetic nerve activity measured in the superior left ganglionic plexus in accordance with an illustrative embodiment.

FIG. 3B depicts that parasympathetic nerve firing frequency was significantly increased with progression of up to approximately 80 days RAP (R=0.6, P<0.05) in accordance with an illustrative embodiment.

FIG. 3C shows examples of parasympathetic nerve activity at baseline and 10 weeks RAP in accordance with an illustrative embodiment.

FIG. 4A is an example of a baseline pre-contrast T1 map in the mapped PLA regions (n=12 dogs at baseline, 14 dogs in persistent AF) in accordance with an illustrative embodiment.

FIG. 4B shows a significant increase of native T1 in the atrium with AF in accordance with an illustrative embodiment.

FIG. 4C depicts that there was also a significant increase in native T2 in the atrium in AF with the increase being progressive over time in accordance with an illustrative embodiment.

FIG. 4D depicts that there was no significant change in ECV with increasing AF in accordance with an illustrative embodiment.

FIG. 5A depicts 8-OHdG staining of stress-damaged nuclei and undamaged nuclei in accordance with an illustrative embodiment.

FIG. 5B depicts an example of tissue with damaged nuclei in accordance with an illustrative embodiment.

FIG. 5C depicts that oxidative damage in 8-oxo-DG staining increased progressively over RAP days in the left atrial regions (R=0.5, P<0.05) in accordance with an illustrative embodiment.

FIG. 5D depicts that the degree of fibrosis increased over RAP days in atrial regions in accordance with an illustrative embodiment.

FIG. 5E depicts that there was a significant positive correlation between DNA oxidative damage and the extent of the degree of dense focal fibrosis (R=0.6, P<0.05) in accordance with an illustrative embodiment.

FIG. 6 depicts a system to measure earlier stage AF progression linked to OS and electrical remodeling in real-time using electrograms (CL, OI, DF, and electrogram voltage), and nerve recordings (peaking at 80 RAP days) in accordance with an illustrative embodiment.

FIG. 7A depicts increased parasympathetic nerve activity (summation nerve signal amplitude) over RAP weeks up to 10 RAP weeks in accordance with an illustrative embodiment.

FIG. 7B depicts that after 10 RAP weeks, there was no clear relationship between parasympathetic nerve firing and the duration of RAP in accordance with an illustrative embodiment.

FIG. 8 depicts that, in the functional MRI analysis, ejection fraction (EF) decreased with a strong correlation coefficient over RAP days (R=0.7, P<0.05) and that stroke volume (SV) also decreased over RAP (R=0.5, p<0.05 in accordance with an illustrative embodiment.

FIG. 9A depicts an example of a myocardium with mainly undamaged nuclei detected with Qupath in accordance with an illustrative embodiment.

FIG. 9B depicts an example of fibro-fatty tissue with dense damaged nuclei close to fibro-fatty region in accordance with an illustrative embodiment.

FIG. 10 depicts increased parasympathetic nerve activity assessed as time between neighbored peaks decreased over RAP weeks (R=0.6, P<0.05) in accordance with an illustrative embodiment.

FIG. 11 depicts a computing system for predicting progression of atrial fibrillation in accordance with an illustrative embodiment.

### DETAILED DESCRIPTION

Atrial fibrillation is the most common sustained cardiac arrhythmia, affecting more than 3 million people in the United States. One in 5 Americans over the age of 65 (estimated 7.6 million) is expected to suffer from AF in 2050. AF leads to a 1.9-fold increased risk of mortality, and a 5-fold higher risk of stroke. Unfortunately, antiarrhythmic drugs and catheter ablation therapies have limited efficacy in the treatment of AF. These limitations of the current treatment approaches have stimulated more interest in the functional, structural, and molecular mechanisms of the genesis and perpetuation and the early quantification of the specific AF progression state to find better treatment approaches for the future. A better understanding and quantification of the time sequences of the specific functional and structural remodeling processes and mechanisms will be critical to the development of earlier and more effective therapies for AF.

In the last two decades, studies in animal models of AF and humans have led to an improved understanding of the molecular underpinnings of the AF disease state. A large number of studies have demonstrated an important role for inflammation, oxidative stress (OS), and autonomic remodeling in the genesis of electrical remodeling (i.e. remodeling of ion channels, gap junctions, and calcium cycling) and eventually, the onset of atrial fibrosis (structural remodeling). In recent years, researchers have shown an important role for increased parasympathetic nerve sprouting (and resulting increase in downstream muscarinic signaling) in the creation of a vulnerable substrate for AF. Recently, the role of NOX2-generated oxidative injury AF in electrical remodeling has been demonstrated, where reactive oxygen species (ROS) lead to PKC-induced ion channel changes underlying refractory period shortening and CAMKII induced changes in Na⁺ channel activity that lead to inhomogeneous conduction and structural remodeling. Despite the contribution of autonomic nerve remodeling, inflammation/OS, and fibrosis to AF, the precise time sequence of these changes in different atrial regions in both atria and the interplay between these mechanisms in AF progression are not well understood.

Several studies have suggested that AF electrogram characteristics reflect underlying AF mechanisms. However, it is unknown which electrogram measures can help define the progression of the AF disease state. The quantification of these risk factors in an earlier AF progression state may delay continuing arrhythmia manifestation, and when treated early reduce the arrhythmia burden in individual patients. Additionally, cardiovascular magnetic resonance imaging (CMR) has emerged as a powerful tool for myocardial tissue characterization. CMR parametric mapping—T1 and T2 relaxation times—have been shown to quantify tissue alterations due to diffuse fibrosis and edema in the ventricular myocardium, but whether these techniques can detect serial changes in atrial myocardium is unknown.

The temporal progression of the arrhythmogenic substrate underlying atrial fibrillation (AF) and the molecular mechanisms underlying this progression to persistent AF are not well understood. The inventor hypothesized that the molecular and structural substrate underlying the temporal progression of AF can be accurately detected by a combination of AF electrogram characteristics and magnetic resonance imaging (MRI) parametric mapping. Described herein are methods and systems that relate to detecting, measuring and/or recording bioelectric signals of the body. More particularly, this application relates to analysis of signals of the heart (e.g., electrical signals, nerve signals, magnet resonance tomography signals) and pathological quantification for diagnostic and treatment purposes of heart diseases like atrial fibrillation.

Experiments were conducted to develop the proposed system. Specifically, AF was induced in 14 dogs (25-35 kg, ≥1 year) by rapid atrial pacing (RAP 3-17 weeks, 600 beats/min), and 8 controls were used. High-resolution epicardial mapping was performed to map 6 atrial regions in both atria using 130 electrodes having an inter-electrode distance of 2.5 mm. In addition, electrogram cycle length (CL), dominant frequency (DF), organization index (OI) and bipolar voltage (V_{bip}) were analyzed. Implantable telemetry recordings (DSI) were used to quantify parasympathetic and sympathetic nerve activity (PNA) and heart rate variability over RAP time. MRI native T1, post-contrast T1, T2 mapping, and extracellular volume fraction (ECV) were assessed using a 1.5 T clinical scanner (Siemens Aera) at baseline and after a prolonged period of persistent AF. In explanted atrial tissue, DNA oxidative damage (8-OHdG staining) and % fibro-fatty tissue were quantified.

As discussed in more detail below, during the experiment the electrogram measures CL, OI decreased (R=0.6, P<0.05), (R=0.5, P<0.05) and DF increased (R=0.4, P<0.05) till 80 days of RAP, but not thereafter when compared to controls. In contrast, bipolar peak-to-peak voltage continued to decrease throughout the duration of RAP. PNA increased post-RAP and plateaued at 80 days. MRI native T1 and T2 times increased with RAP days (R=0.5, P<0.05), (R=0.6, P<0.05) in PLA throughout the duration of RAP. Increased RAP days correlated with increasing 8-OHdG and with the extent of fibrosis (R=0.5, P<0.05 for both). Based on these results, it has been found that a combination of AF electrogram characteristics and T1/T2 MRI can help accurately detect not only early-stage AF remodeling (secondary to autonomic remodeling and inflammation-related changes such as oxidative stress (OS)), but also more advanced AF remodeling due to increasing OS and fibrosis.

The inventor has hypothesized that AF progression resulting from increasing autonomic remodeling, oxidative stress, and fibrosis can be uncovered in real time by using a combination of AF electrogram characteristics and T1/T2 on MRI mapping. Specifically, the following considerations were addressed in developing the proposed methods and systems: i) What is the precise time sequence of changes in AF electrogram characteristics (both frequency and amplitude characteristics) in the setting of progressively increasing duration of AF in a canine rapid atrial pacing (RAP) model?, ii) Are electrical and structural remodeling in the atria reflected on T1/T2 cardiovascular magnetic resonance imaging in the RAP model?, and iii) Do changes in AF electrogram characteristics and MR imaging with increasing duration of AF reflect progressive changes in autonomic nerve remodeling (firing), inflammation/OS, and fibrosis? To help determine answers to these questions, the inventor conducted experiments as noted above.

In the primary experiment, rapid atrial pacing (RAP) was used to induce AF in 14 dogs for 3-14 weeks. Before all the procedures, animals were premedicated with acepromazine (0.01-0.02 mg/kg, Vedco) and induced with propofol (3-7 mg/kg, Zoetis). All experiments were performed under general anesthesia (inhaled) with isoflurane (1-3%). For pacemaker insertion, the right jugular vein was accessed by direct cutdown and ligated distally. A bipolar screw-in Medtronic pacing lead was inserted through an incision in the right jugular vein. The tip of the lead was fluoroscopically placed and fixed in the RA appendage after confirming an adequate capture threshold (<0.5 mV with a pulse width of 0.4 ms). The proximal end of the pacing lead was connected to a custom-modified Medtronic programmable pulse generator that was subsequently implanted in a subcutaneous pocket in the neck.

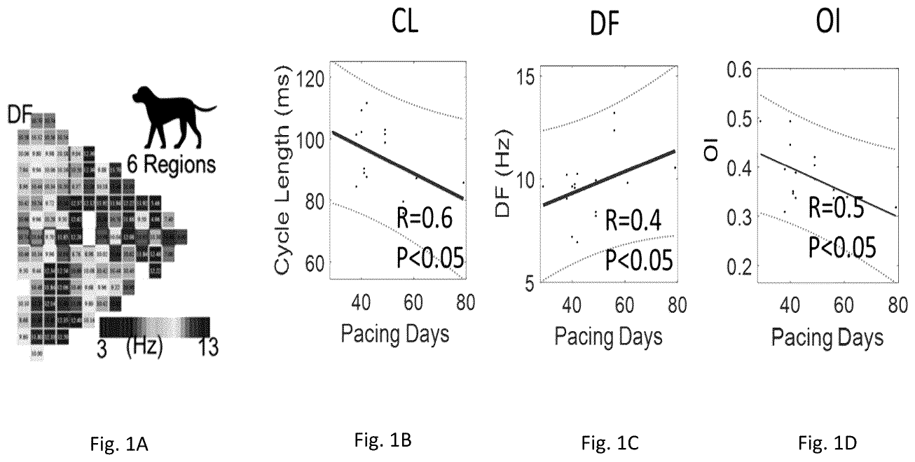

As part of the experiment, the inventor performed high-density open chest mapping on the epicardium after the induction of persistent AF using the UnEmap mapping system. Six atrial regions were mapped in both atria in all 14 AF dogs. The UnEmap mapping system (University of Auckland, Auckland, New Zealand) includes a triangular mapping plaque and records 117 bipolar electrogram signals (1 kHz sampling rate, 130 electrodes, interelectrode distance of 2.5 mm). In alternative embodiments, a different mapping system and/or technique may be used. FIG. 1A depicts a high resolution mapping plaque in accordance with an illustrative embodiment.

Bipolar electrograms were recorded from 6 different regions in both atria: posterior left atrium (PLA), left atrial free wall (LAFW), left atrial appendage (LAA), posterior right atrium (PRA), right atrial free wall (RAFW), right atrial appendage (RAA). Various AF signal characteristics were analyzed, including dominant frequency (DF), cycle length (CL), and organization index (OI). The inventor further analyzed sub-region sizes in the percentage of the mapping field with >10% change in the DF delta maps and measured the effective refractory period. FIG. 1B depicts an electrogram measure of cycle length (dependent on pacing days in the posterior left atrium) in accordance with an illustrative embodiment. FIG. 1C depicts an electrogram measure of dominant frequency (dependent on pacing days in the posterior left atrium) in accordance with an illustrative embodiment. FIG. 1D depicts an electrogram measure of organization index (dependent on pacing days in the posterior left atrium) in accordance with an illustrative embodiment.

The dominant frequency (DF) has been shown to correspond to rotational activity in AF. The inventor calculated the dominant frequency with the highest power in the power spectrum using the fast Fourier transform. Bandpass filtering with cutoff frequencies of 40 and 250 Hz were used during the analysis. The organization index (OI) is a frequency domain parameter of the temporal organization or regularity. The OI was computed as the area under 1-Hz windows of the DF peak and the next 3 harmonic peaks divided by the total area of the spectrum from 3 Hz up to the fifth harmonic peak. The bipolar voltage is assessed as peak-to-peak voltage in the bipolar signals.

In addition, during the mapping, data was collected with the GE Prucka Cardiolab system (GE Healthcare) and 2 rectangular mapping plaques with 12 electrodes (3×4 electrodes, interelectrode distance 5 mm). The inventor excluded electrodes (<10%) because of inadequate quality due to noise or poor contact.

The inventor also recorded parasympathetic nerve recordings (DSI Harvard Bioscience Inc.) in the superior left ganglionic plexi (SLGP) recordings from the stellate ganglion in 3 dogs weekly over a RAP time of 11-12 weeks. This was done through placement of a fully implanted telemetry device to wirelessly store and transmit nerve recordings. Pairs of bipolar electrodes were placed at the level of the cardiac nerves/ganglia (e.g., stellate ganglion) and affixed with silk suture. Similarly, electrodes were placed at the cardiac base/apex to permit ECG recordings. The leads were surgically routed through the ventral aspect of the rib space, and a subcutaneous pocket was formed caudal to the thoracotomy incision to contain the device. Excess lead length and the telemetry unit were secured into the pocket and closed. Data were recorded weekly during RAP for over 3-4 hours in real-time at a sampling rate of 1,000 samples per second per channel and then analyzed offline. The data from stellate ganglion (channel 2) and superior left ganglionic plexi (channel 1) were high-pass filtered (150 Hz) and low-pass filtered (1000 Hz). To quantify the high-frequency discharges associated with nerve activity, the inventor integrated the amplitude of nerve activity over time. Additionally, the inventor assessed parasympathetic nerve activity as the times between parasympathetic (PNA) spikes.

In addition, MRI scans were performed on a 1.5 T clinical scanner (MAGNETOM Aera; Siemens Healthcare, Erlangen, Germany). The CMR scan protocol included cine imaging, native T1 mapping, and post-contrast T1 mapping. Cine imaging utilized a segmented retrospectively-gated steady-state free-precession (SSFP) sequence performed in a short axis stack spanning the left ventricle and left atrium, along with 2-chamber, 3-chamber, and 4-chamber views. Native T1 mapping utilized a modified look locker inversion-recovery (MOLLI) sequence with a 5 (3) 3 sampling pattern performed in the 3-chamber, mid-left atrial short axis, and mid-left ventricular short axis planes. T2 mapping was performed by the acquisition of 3 successive T2-prepared SSFP images with varying T2 prep times, performed in the same imaging planes as native T1 mapping. Post-contrast T1 mapping utilized a MOLLI sequence with 4 (1) 3 (1) 2 sampling pattern performed in the same imaging planes as native T1 mapping more than 10 minutes after the intravenous administration of gadolinium-based contrast agent (Gd-DTPA, 0.2 mmol/kg). The T1 and T2 mapping voxel size was 1.0×1.0×5 mm. Cine images were quantitatively analyzed using CVI42 (Circle) for left ventricular end-diastolic volume, end-systolic volume, stroke volume, and ejection fraction.

The T1 and T2 maps were analyzed using QMass MR 7.6 (Medis, Leiden, The Netherlands) by drawing regions of interest (ROI) in the posterior left atrium on the 3-chamber and LA mid-short axis views, and the inferolateral LV myocardium on the 3-chamber view. Care was taken to draw ROI's only in segments of the LA that were thick enough to avoid signal averaging with the blood pool. Mapping analysis was performed by an analyzer with >15 years of CMR analysis experience (BCB) who was blinded to the number of pacing days. Extracellular volume fraction was calculated as ECV=ΔR1myocardium/ΔR1bloodpool (1−hematocrit), where R1=1/T1 and ΔR1 is post-contrast−pre-contrast R1. Left atrial T1, T2, and ECV values from the 3-chamber and LA short-axis views were averaged for the final analysis.

Tissue analysis was also performed. The tissue sections from the 6 different atrial tissue regions, the level of oxidative stress, and the degree of fibrosis were analyzed in 6 animals. Linear regression analysis was performed to assess the correlation between the tissue section characteristics of oxidative stress level and the degree of fibrosis and electrogram (EGM) characteristics. After confirming a deep plane of anesthesia, the heart was excised and immersed it in ice-cold cardioplegia solution containing (mmol/l) NaCl 128, KCl 15, HEPES 10, MgSO₄ 1.2, NaH₂PO₄ 0.6, CaCl₂) 1.0, glucose 10, and heparin (0.0001 U/ml); pH 7.4. All solutions were equilibrated with 100% O₂. The hearts were cannulated via the aorta and perfused with ice-cold cardioplegia solution containing protease inhibitors (Millipore Sigma, P8340) until the vessels were clear of blood, and the tissue was cold. Atrial tissue was excised and the six atrial regions were dissected. The preparations were frozen in OCT tissue freezing medium (VWR Chemicals) at ˜−50° C. in 2-methyl butane cooled by dry ice and stored at −80° C. until use. Alternatively, samples were fixed in 10% formalin and embedded in paraffin.

The frozen preparations were serially sectioned (at −25° C.) at 10 μm thickness. Frozen sections were mounted on Superfrost Plus slides (VWR) and stored at −80° C. until use. The sections were taken from a −80° C. freezer, air-dried, underwent fixation with 75% acetone/25% ethanol, and washed 3 times in TBS-T. The sections were then treated in 3% hydrogen peroxide. After washing three times in TBS-T, the sections were blocked in protein block reagent (Dako) and then incubated with primary antibody against 8-OHdG (mouse monoclonal antibody; JalCA, MOG-020P) diluted in antibody diluent reagent (Dako) in a humid box at −4° C. overnight. The sections were washed three times in TBS-T and incubated with Dako envision secondary antibody in a humid box at RT for 30 min. After washing three times in TBS-T, the sections were dehydrated with a series of Ethanol and Xylene and mounted with cytosol (VWR).

Paraffin sections with 5 μm thickness were stained using Masson's Trichrome stain kit (Sigma). In brief, paraffin was removed in xylene for three minutes twice and then in the mixture of 100% xylene and absolute ethanol for three minutes twice. Sections were then rehydrated with ethanol series which include absolute ethanol (twice), 95% ethanol, 70% ethanol, and 50% ethanol. The paraffin sections were treated with Bouin's mordant at room temperature overnight. The following day the sections were rinsed in running water to remove the excess yellow. The sections were stained in Weigert's Iron Hematoxylin Solution for 5 minutes. Next, it was washed under running water for 5 minutes and briefly rinsed in distilled water. The sections were then stained in Beibrich Scarlet-Acid fuchsin for 5 minutes, followed by a rinse in distilled water. Subsequently, the sections were incubated in the phosphomolybdic-phosphotungstic acid solution for 5 minutes. The section of interest was then stained in Aniline Blue solution for 5 minutes. The sections were incubated in 1% Glacial acetic acid for 2 minutes. The sections were then dehydrated through an ethanol series, which included 70%, 90%, and absolute ethanol (twice). Then, the sections were placed in xylene for 5 minutes twice. A coverslip was finally placed using cytosol mounting media on the sections for microscopic examination.

The inventor examined the levels of 8-OHdG—a biomarker for oxidative damage of DNA—in atrial tissues. The histological tissue sections were digitalized (20×) using the TissueFax system (TissueGnostics), and the ratio of oxidatively damaged nuclei (8-OHdG stained nuclei) against the total number of nuclei was calculated using the digital pathology software Qupath. Alternatively, different software and/or techniques can be used. To examine the degree of fibrosis, Masson's trichrome-stained tissue sections were digitalized (20×) and for the quantitative morphometric analysis, the sections were analyzed using ImageJ software.

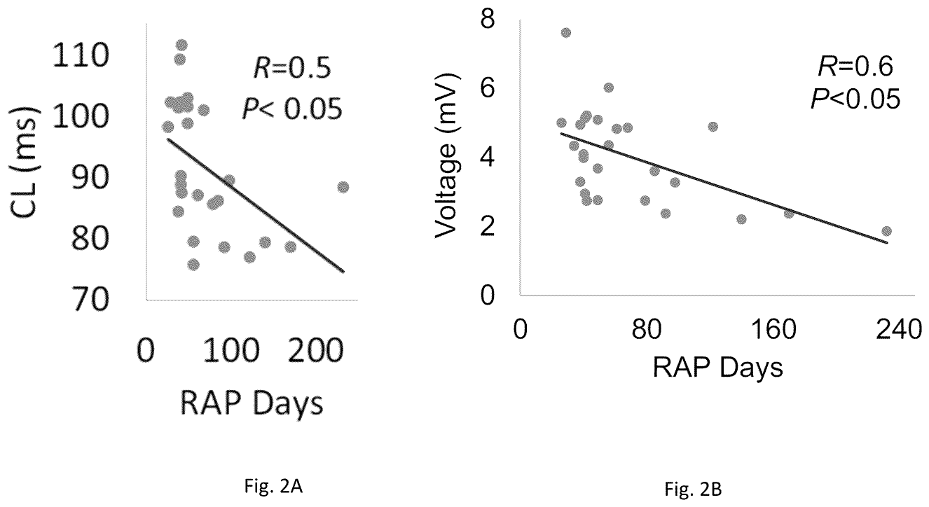

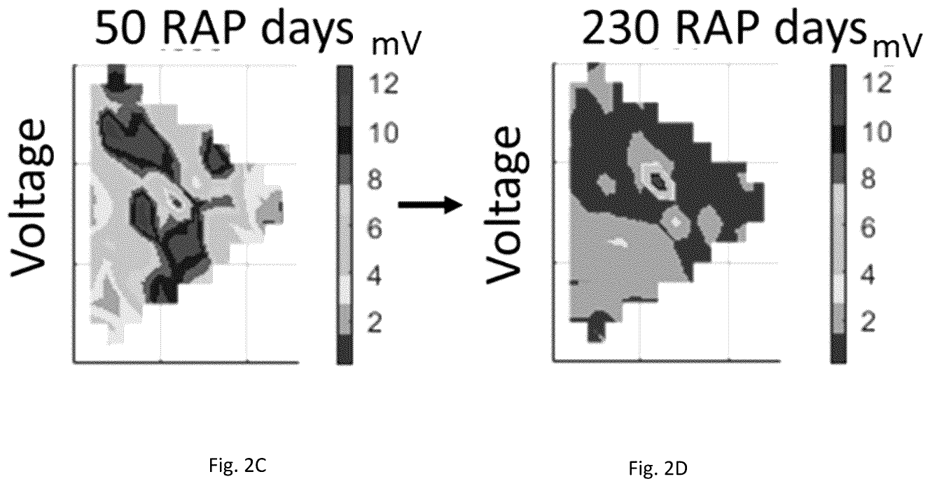

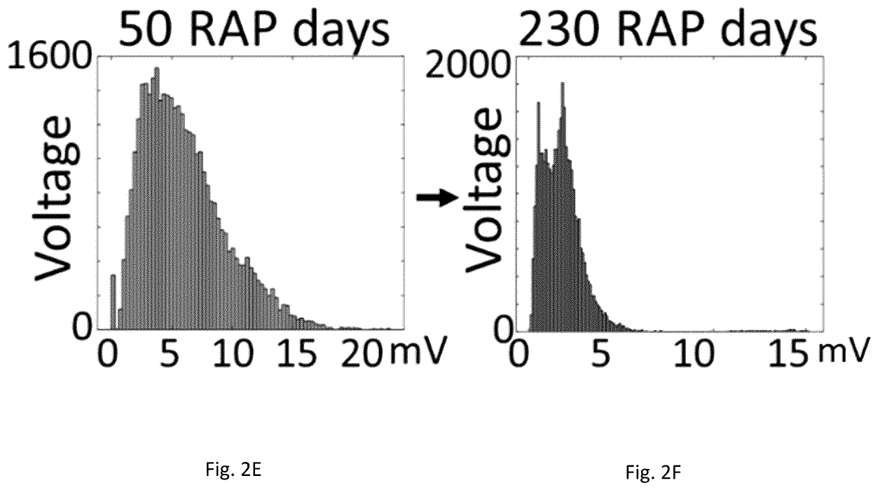

Early versus late atrial remodeling is characterized by unique and distinctive electrogram characteristics. To assess if established electrogram measures can identify the current AF progression state a correlation analysis of the electrogram measures CL, OI, and DF was performed over rapid atrial pacing time. It was found that the electrogram measures CL, OI decreased (R=0.6, P<0.05), (R=0.5, P<0.05) and DF increased (R=0.4, P<0.05) over time of RAP, indicating increasing frequency and disorganization of AF, as shown in FIGS. 1B-1D. However, after 80 days of RAP, no further changes in the EGM CL, OI, and DF were observed. FIG. 2A shows CL changes in the development of persistent AF dependent on pacing days in accordance with an illustrative embodiment. Importantly after 80 RAP days, the CL plateaued with no additional changes. In contrast, FIG. 2B depicts that bipolar voltage (V_{bip}) decreased strongly over the number of RAP days (R=0.7, P<0.05) in the PLA not only pre- but also post-80 RAP days in accordance with an illustrative embodiment. FIG. 2C depicts example V_{bip} maps at 50 RAP days in accordance with an illustrative embodiment. FIG. 2D depicts example V_{bip} maps at 230 RAP days in accordance with an illustrative embodiment. FIG. 2E is a histogram corresponding to FIG. 2C that shows the decrease in V_{bip} over RAP days in accordance with an illustrative embodiment. FIG. 2F is a histogram corresponding to FIG. 2D that shows the decrease in V_{bip} over RAP days in accordance with an illustrative embodiment.

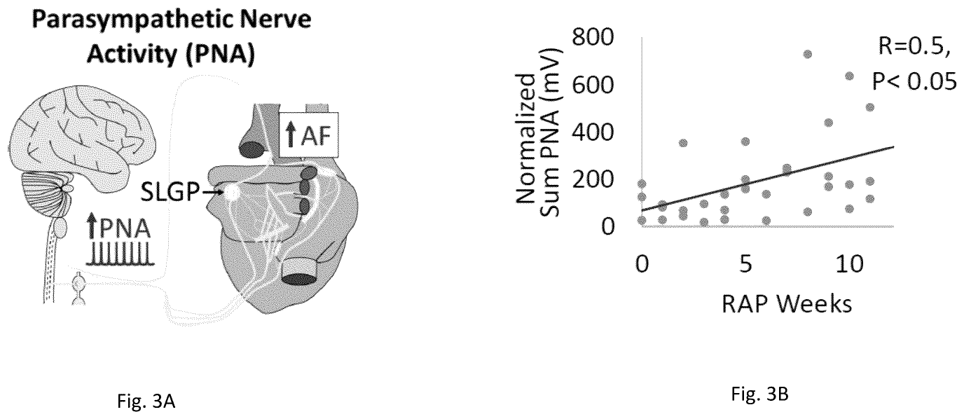

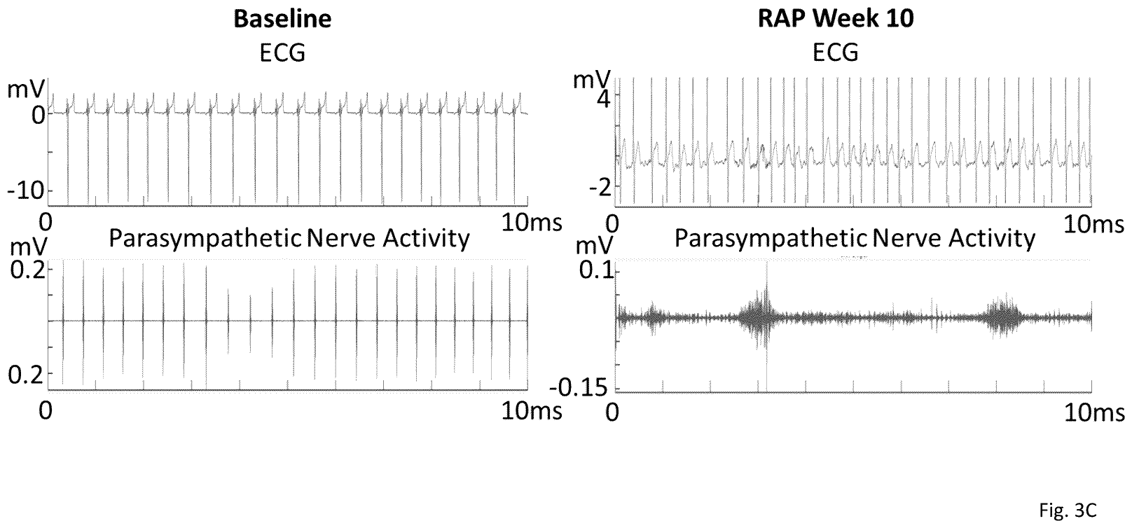

It was also determined that progression of AF is accompanied by a progressive increase in the frequency of parasympathetic nerve firing. Autonomic nerve activity (firing) plays a major role in AF remodeling, with the AF disease state shown to correlate with increased parasympathetic nerve firing. Parasympathetic nerve remodeling and firing have been thought to play a particularly important role in electrical remodeling in AF. However, the temporal changes in nerve firing that occur with progressively increasing duration of AF are not well understood. Therefore, the inventor systematically assessed parasympathetic nerve firing from the superior left ganglionic plexus and stellate ganglia. Specifically, parasympathetic nerve frequency characteristics were examined over RAP days. FIG. 3A shows a schematic of parasympathetic nerve activity measured in the superior left ganglionic plexus in accordance with an illustrative embodiment. FIG. 3B depicts that parasympathetic nerve firing frequency was significantly increased with progression of up to approximately 80 days RAP (R=0.6, P<0.05) in accordance with an illustrative embodiment. FIG. 3C shows examples of parasympathetic nerve activity at baseline and 10 weeks RAP in accordance with an illustrative embodiment.

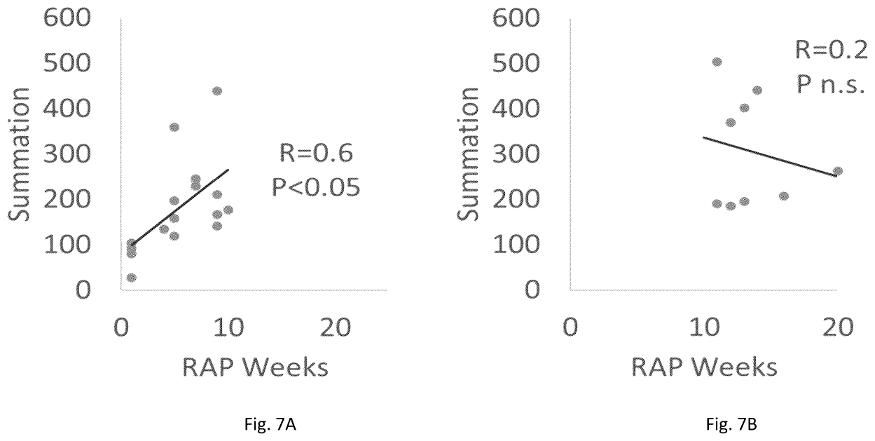

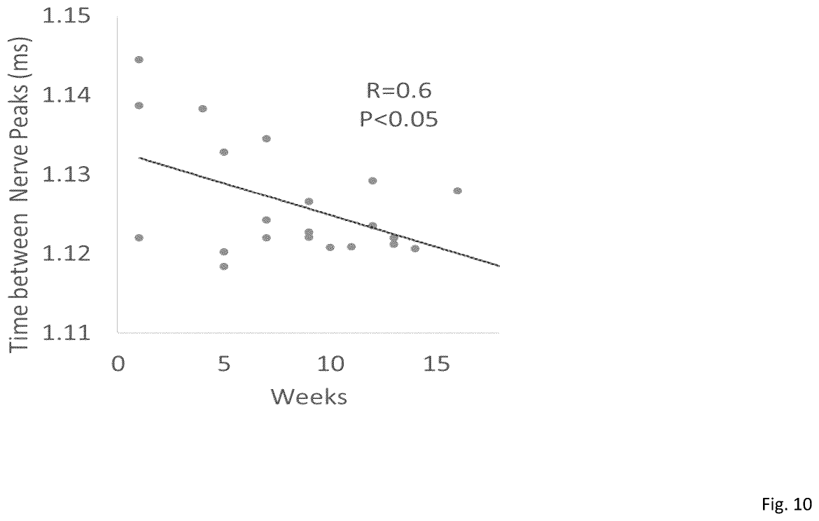

These results are also illustrated in FIG. 7. Specifically, FIG. 7A depicts increased parasympathetic nerve activity (summation nerve signal amplitude) over RAP weeks up to 10 RAP weeks in accordance with an illustrative embodiment. FIG. 7B depicts that after 10 RAP weeks, there was no clear relationship between parasympathetic nerve firing and the duration of RAP in accordance with an illustrative embodiment. FIG. 10 depicts increased parasympathetic nerve activity assessed as time between neighbored peaks decreased over RAP weeks (R=0.6, P<0.05) in accordance with an illustrative embodiment.

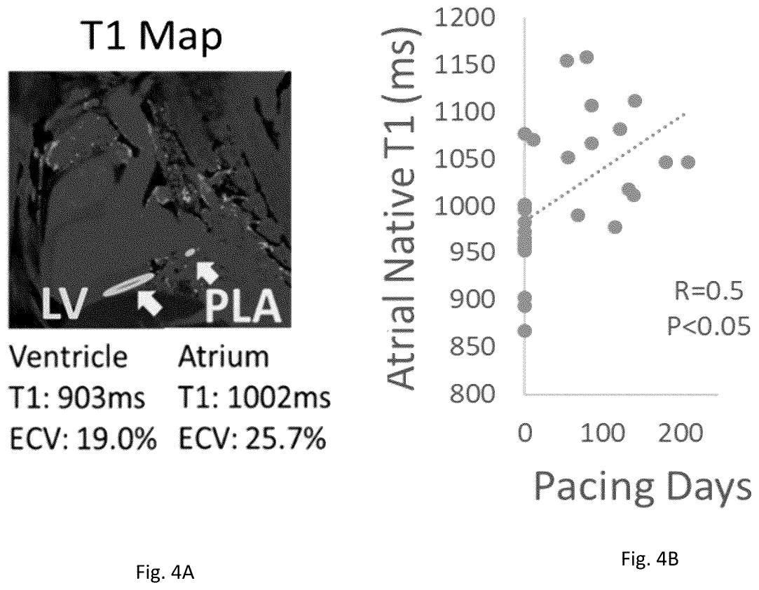

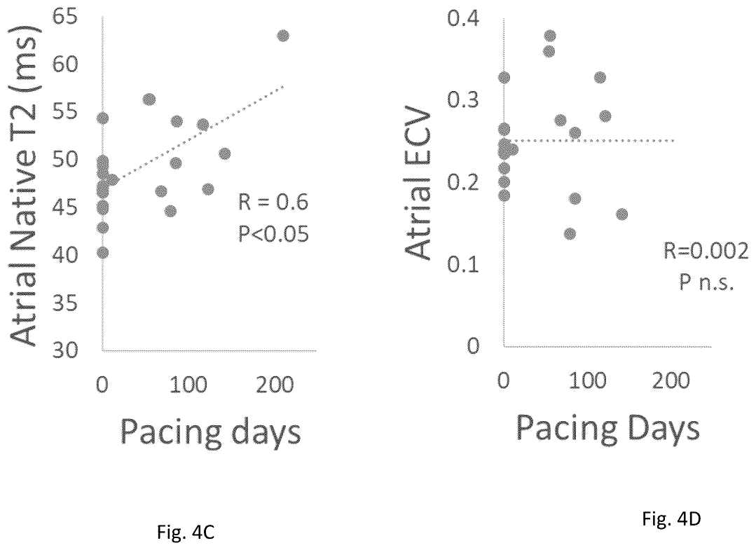

It was also found that the increasing duration of AF is accompanied by a progressive increase in T1 and T2 on cardiac MR imaging. Although the AF disease state is accompanied by increased atrial inflammation, edema, and fibrosis, only recently have such changes been examined with cardiac imaging. As far as is known, this is the first study to systematically use T1/T2 mapping and ECV to examine atrial remodeling in real-time with increasing duration of AF. FIG. 4A is an example of a baseline pre-contrast T1 map in the mapped PLA regions (n=12 dogs at baseline, 14 dogs in persistent AF) in accordance with an illustrative embodiment. FIG. 4B shows a significant increase of native T1 in the atrium with AF in accordance with an illustrative embodiment. Furthermore, this increase in T1 was progressive with increasing duration of RAP. FIG. 4C depicts that there was also a significant increase in native T2 in the atrium in AF with the increase being progressive over time in accordance with an illustrative embodiment. FIG. 4D depicts that there was no significant change in ECV with increasing AF in accordance with an illustrative embodiment. Collectively, the inventor noted a significant increase in T1 and T2 with increasing duration of AF in the RAP model, but without an accompanying increase in ECV.

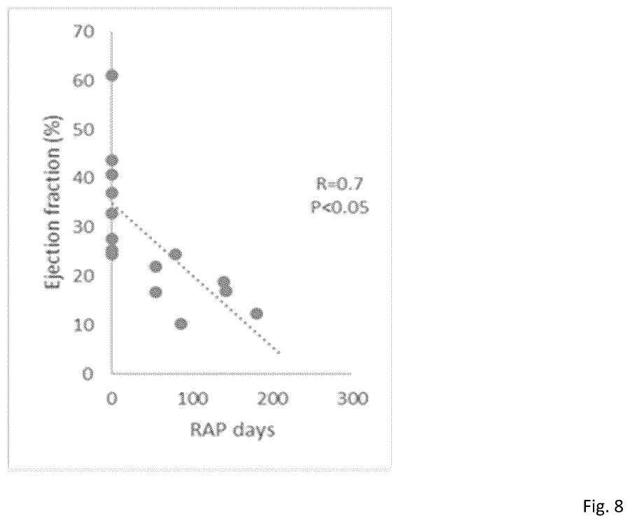

The experimental data also showed a pattern of decreasing ejection fraction and stroke volume over RAP days. FIG. 8 depicts that, in the functional MRI analysis, ejection fraction (EF) decreased with a strong correlation coefficient over RAP days (R=0.7, P<0.05) and that stroke volume (SV) also decreased over RAP (R=0.5, p<0.05 in accordance with an illustrative embodiment.

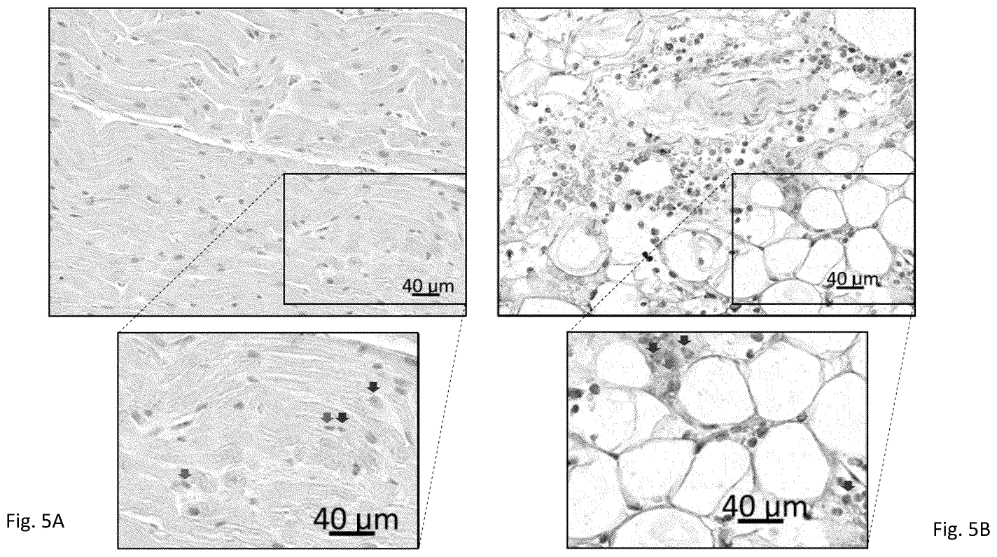

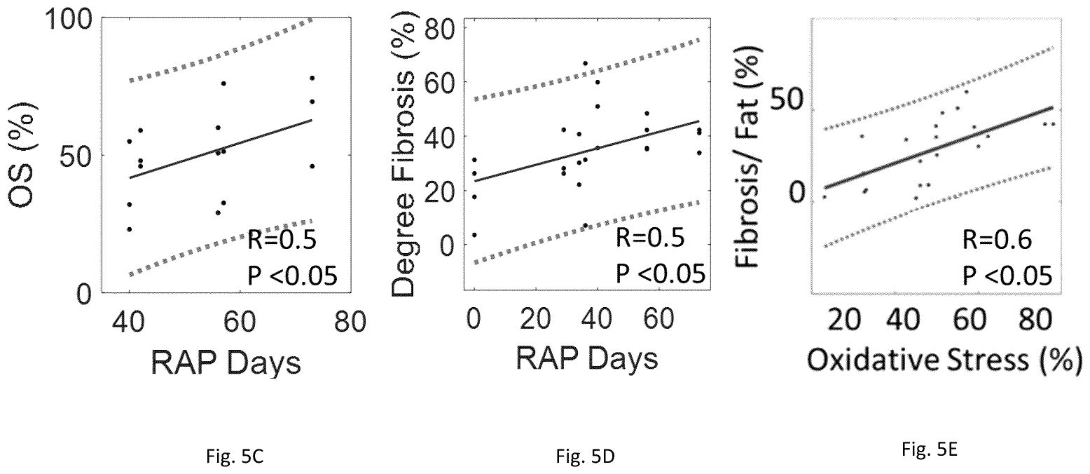

It was further found that the progression of AF is accompanied by a progressive increase in DNA oxidative damage and fibro-fatty infiltration. RAP-induced AF led to a significant increase in fibrofatty infiltration in the atrium, with fibrosis increasing progressively over RAP days in atrial regions. The inventor quantified oxidative stress (OS) levels in persistent AF by examining DNA oxidative damage (using 8-oxo-DG staining). AF progression induced by RAP led to a significant increase in DNA oxidative damage. This increase in oxidative damage was noted in atrial myocytes as well as in regions of fibrofatty infiltration. FIG. 5A depicts 8-OHdG staining of stress-damaged nuclei and undamaged nuclei in accordance with an illustrative embodiment. FIG. 5B depicts an example of tissue with damaged nuclei in accordance with an illustrative embodiment. FIG. 5C depicts that oxidative damage in 8-oxo-DG staining increased progressively over RAP days in the left atrial regions (R=0.5, P<0.05) in accordance with an illustrative embodiment. FIG. 5D depicts that the degree of fibrosis increased over RAP days in atrial regions in accordance with an illustrative embodiment. Of note, FIG. 5E depicts that there was a significant positive correlation between DNA oxidative damage and the extent of the degree of dense focal fibrosis (R=0.6, P<0.05) in accordance with an illustrative embodiment.

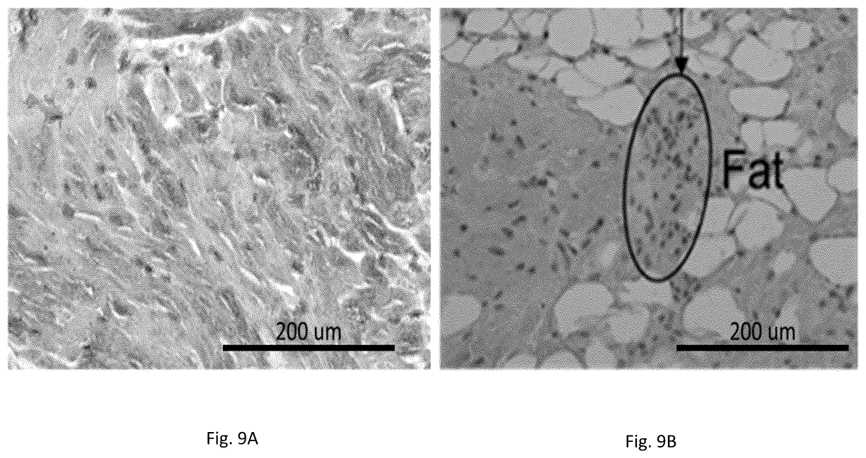

These results are also illustrated in FIG. 9. Specifically, FIG. 9A depicts an example of a myocardium with mainly undamaged nuclei detected with Qupath in accordance with an illustrative embodiment. FIG. 9B depicts an example of fibro-fatty tissue with dense damaged nuclei close to fibro-fatty region in accordance with an illustrative embodiment. To distinguish, oxidative stress-damaged nuclei can be stained in a first color (e.g., brown), and undamaged nuclei are stained in a second color (e.g., blue).

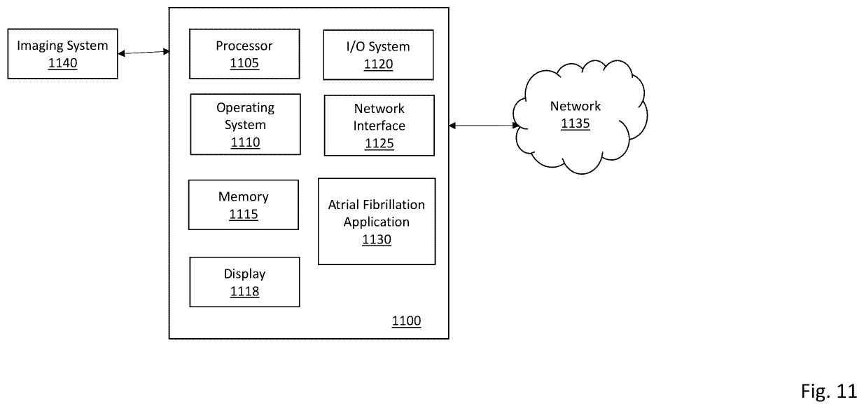

In one embodiment, the proposed system can include a computing system and/or computing components. The computing system can be in the form of a dedicated computing system, a personal computing device (e.g., smartphone), a laptop computer, desktop computer, etc. FIG. 11 depicts a computing system 1100 for predicting progression of atrial fibrillation in accordance with an illustrative embodiment. In one embodiment, at least a portion of the computing system 1100 can be remote from the imaging system(s) and/or electrodes, but in communication therewith through a network 1135 or other form of wireless communication.

The computing system 1100 includes a processor 1105, an operating system 1110, a memory 1115, a display 1118, an input/output (I/O) system 1120, a network interface 1125, and an atrial fibrillation application 1130. In alternative embodiments, the computing system 1100 may include fewer, additional, and/or different components. The components of the computing system 1100 communicate with one another via one or more buses or any other interconnect system. As discussed, the computing system 1100 can be any type of computing system (e.g., smartphone, tablet, laptop, desktop, etc.), including a dedicated standalone computing system that is designed to perform the atrial fibrillation analysis and prediction. In one embodiment, at least a portion of the computing system 1100 may be incorporated into an imaging system 1140 (e.g., MRI, CAT, etc.), and can be in communication with an electrode array mounted to a patient.

The processor 1105 can be in electrical communication with and used to control any of the system components described herein. For example, the processor 1105 can be used to execute the atrial fibrillation application 1130, control the imaging system 1140, process image data, obtain and process electrode data, control a pacemaker, generate and/or control a pacing signal, etc. The processor 1105 can be any type of computer processor known in the art and can include a plurality of processors and/or a plurality of processing cores. The processor 1105 can include a controller, a microcontroller, an audio processor, a graphics processing unit, a hardware accelerator, a digital signal processor, etc. Additionally, the processor 1105 may be implemented as a complex instruction set computer processor, a reduced instruction set computer processor, an x86 instruction set computer processor, etc. The processor 1105 is used to run the operating system 1110, which can be any type of operating system.

The operating system 1110 is stored in the memory 1115, which is also used to store programs, received image data, received electrode data, pacing data, network and communications data, peripheral component data, the atrial fibrillation application 1130, and other operating instructions. The memory 1115 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 1115 can be in the cloud to provide cloud storage for the system. Similarly, in one embodiment, any of the computing components described herein (e.g., the processor 1105, etc.) can be implemented in the cloud such that the system can be run and controlled through cloud computing.

The I/O system 1120 is the framework which enables users and peripheral devices to interact with the computing system 1100. The display 1118 can include a touch screen in some embodiments, and the touch screen can be part of the I/O system 1120 that allows a user to make selections, control sub-systems, view results, etc. The display 1118 can be any type of display, including a monitor, projector, etc., and can be used to present user interface screens, control screens, captured images, captured video, and other data to the user. The I/O system 1120 can also include one or more speakers, one or more microphones, a keyboard, a mouse, one or more buttons or other controls, etc. that allow the user to interact with and control the computing system 1100. The I/O system 1120 also includes circuitry and a bus structure to interface with peripheral computing devices such as the imaging system, electrodes, power sources, universal service bus (USB) devices, data acquisition cards, peripheral component interconnect express (PCIe) devices, serial advanced technology attachment (SATA) devices, high-definition multimedia interface (HDMI) devices, proprietary connection devices, etc.

The network interface 1125 includes transceiver circuitry (e.g., a transmitter and a receiver) that allows the computing system 1100 to transmit and receive data to/from other devices such as remote computing systems, servers, websites, imaging systems, electrode arrays, etc. The network interface 1125 enables communication through the network 1135, which can be one or more communication networks. The network 1135 can include a cable network, a fiber network, a cellular network, a wi-fi network, a landline telephone network, a microwave network, a satellite network, etc. The network interface 1125 also includes circuitry to allow device-to-device communication such as Bluetooth® communication.

The atrial fibrillation application 1130 can include software and algorithms in the form of computer-readable instructions which, upon execution by the processor 1105, performs any of the various operations described herein such as controlling the imaging system 1140 to capture images, analyzing the captured images, altering settings of the imaging system 1140, analyzing the collected electrode data, controlling the electrode array, generating maps, making predictions of the likely progression of atrial fibrillation based on the analysis, generating a warning if the analysis identifies a serious problem with the patient, etc.

The atrial fibrillation application 1130 can also utilize the processor of the computing system to perform additional tasks such as identifying and predicting the specific atrial fibrillation (AF) progression state resulting from increasing autonomic remodeling, oxidative stress, and fibrosis in real time by using a combination of AF electrogram characteristics, parasympathetic and sympathetic nerve recordings, and T1/T2 on MRI mapping and/or additional input signals in individual patients. The atrial fibrillation application 1130 can predict based on the current AF progression state the next progression states (molecular including oxidative stress levels), functional characteristics (cycle length, organization index, dominant frequency, voltage in electrogram characteristics), nervous system related characteristics (parasympathetic, sympathetic nerve signal characteristics), structural characteristics (degree of fibrosis, degree of fat), risk factors, and survival rate in the next weeks/months/years if the patient will not receive a treatment. The atrial fibrillation application 1130 can also predict based on the current AF progression state the next progression states the optimal treatment plan for the next weeks/months/years if the patient will receive a treatment.

In another embodiment, the atrial fibrillation application 1130 predicts, based on the current AF progression state, AF termination to normal sinus rhythm, duration of normal sinus rhythm, duration of AF episodes, the next AF progression state(s), risk factors and survival rate in the next weeks/months/years if the patients will receive a specific or optimal treatment. In one embodiment, the atrial fibrillation application predicts early and/or late AF progression state in real time based on high resolution electrogram characteristics including cycle length, organization index, dominant frequency, and voltage. In another embodiment, the atrial fibrillation application 1130 predicts AF progression state based on parasympathetic and sympathetic nerve signal characteristics using telemetry. In another embodiment, the atrial fibrillation application predicts AF progression state based on T1/T2/ECV in magnet resonance tomography imaging. In another embodiment, the atrial fibrillation application 1130 predicts AF progression state based on oxidative stress levels in the heart tissue in left and right atrial sub-regions. In another embodiment, the atrial fibrillation application 1130 predicts AF progression state based on fibrosis and fat percentage in the heart tissue in left and right atrial sub-regions.

In one embodiment, the atrial fibrillation application 1130 quantifies nerve frequency as the time between nerve peaks assessed with zero-crossings, the number of nerve signals crossing zero mV, and the variability of zero-crossings. In another embodiment, the atrial fibrillation application 1130 quantifies nerve frequency as the area under the nerve signal and/or the variability of the area under the nerve curve. In another embodiment, the atrial fibrillation application 1130 quantifies nerve activity frequency assessed with heart rate and heart rate variability.

In another embodiment, the atrial fibrillation application 1130 synchronously quantifies intracardiac electrograms, body surface electrograms, nerve activity, and frequency assessed in real-time at baseline, during the catheter ablation or drug or gene treatment or nerve stimulation to detect changes in AF progression or termination of AF to normal sinus rhythm or atrial flutter. In another embodiment, the atrial fibrillation application 1130 synchronously quantifies fractionated electrograms in AF and or sinus rhythm in intracardiac electrograms, body surface electrograms, nerve activity, and frequency assessed in real-time at baseline, during the catheter ablation, to detect parasympathetic and sympathetic responses during AF ablation and association with the presence of pre- and post-ablation fractionated electrograms in sinus rhythm, atrial fibrillation, and atrial flutter. In another embodiment, the atrial fibrillation application 1130 synchronously quantifies fractionated electrograms in AF and/or sinus rhythm in intracardiac electrograms, body surface electrograms, nerve activity, and frequency assessed in real-time at baseline, pre and post drug treatment with N-acetyl cysteine or another drug to detect parasympathetic and sympathetic responses during AF ablation and association with the presence of pre- and post-ablation fractionated electrograms in sinus rhythm, atrial fibrillation, and atrial flutter. In another embodiment, the atrial fibrillation application 1130 synchronously quantifies fractionated electrograms in AF and/or sinus rhythm in intracardiac electrograms, body surface electrograms, nerve activity, and frequency assessed in real-time at baseline, pre and post gene treatment with knock-down of NOX2 or another drug to detect parasympathetic and sympathetic responses during AF ablation and association with the presence of pre- and post-ablation fractionated electrograms in sinus rhythm, atrial fibrillation, and atrial flutter.

In another embodiment, the atrial fibrillation application 1130 uses artificial intelligence algorithms, electrograms, and nerve recordings on the body skin to detect and predict AF progression state. In another embodiment, the atrial fibrillation application 1130 uses a digital twin with multimodal input parameters including numerical field simulation, artificial intelligence algorithms, electrograms, nerve recordings on the body skin, MRI, and other parameters to simulate and predict AF progression state, the optimal treatment, the next AF progression states, AF recurrence, and survival rate. The atrial fibrillation application 1130 can utilize the processor 1105 and/or the memory 1115 and/or the display 1118 as discussed above. In an alternative implementation, the atrial fibrillation application 1130 can be remote or independent from the computing device 1100, but in communication therewith.

Thus, in summary, to develop the present system, the inventor has systematically studied the real-time progression of a vulnerable AF substrate by using a combination of AF electrogram characteristics and newer MRI-based techniques that are thought to reflect atrial edema (due to inflammation) and fibrosis. The inventor also systematically examined the temporal sequence of changes in parasympathetic nerve firing, DNA oxidative damage, and fibrosis that occur in progressive AF, and correlated these with real-time electrogram and MRI changes.

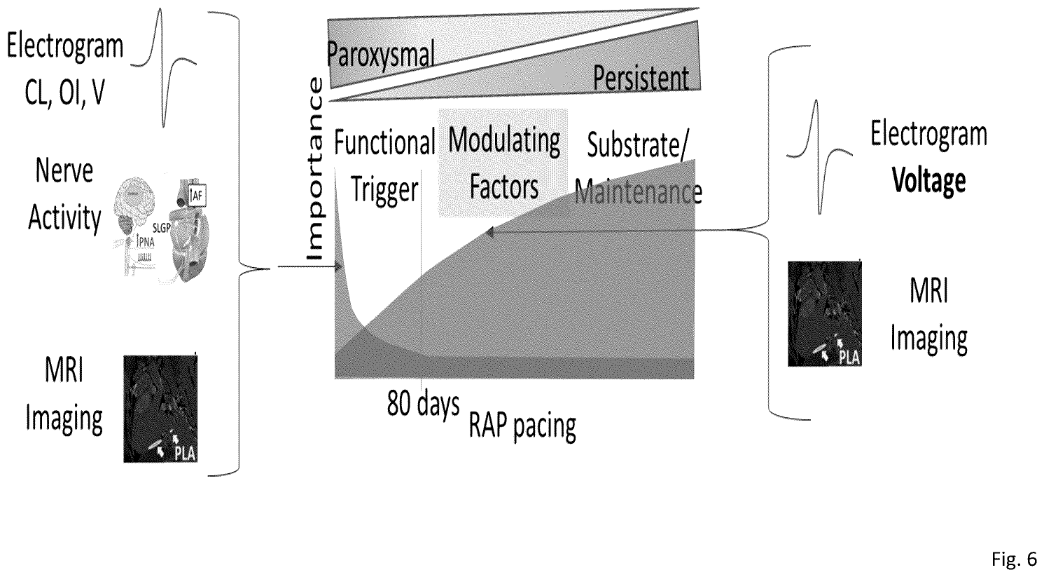

It was found that AF electrogram changes accurately reflect both early and late stages of AF progression—the role of autonomic remodeling, oxidative injury, and fibrosis. An important finding of the study is that while frequency measures of AF electrograms—CL, OI, and DF—are closely correlated with AF progression, this correlation is only accurate in the earlier stages of AF remodeling (up to 80 days of RAP), with EGM measures tending to plateau thereafter. FIG. 6 depicts a system to measure earlier stage AF progression linked to OS and electrical remodeling in real-time using electrograms (CL, OI, DF, and electrogram voltage), and nerve recordings (peaking at 80 RAP days) in accordance with an illustrative embodiment. Longer-term remodeling (post 80 RAP days) linked to fibrosis can be identified using the electrogram voltage. In some embodiments, MRI imaging including T1 mapping may be complementary to detecting AF remodeling states.

A similar pattern of progression was noted for parasympathetic nerve firing, with left ganglionated plexi nerve frequency also tending to plateau at 80 RAP days. These data suggest that frequency measures of AF such as CL, OI, and DF may at least in part reflect autonomic remodeling in the fibrillating atrium. Indeed, a complex interplay between the sympathetic and parasympathetic nervous system in AF remodeling has been demonstrated. Parasympathetic nerve activity shortens the effective refractory period in the atria in an inhomogeneous fashion and can affect conduction and contractility. It has been shown that region-specific parasympathetic nerve remodeling in the left atrium contributes to the creation of a vulnerable substrate for AF. The close temporal relationship between AF frequency characteristics (CL, OI, DF) and parasympathetic nerve firing (increase in parasympathetic nerve frequency and decrease in nerve signal amplitude recorded from the superior left GP) strongly suggests a role for parasympathetic nerve firing in early-stage AF remodeling (<80 RAP days). The current data are also consistent with earlier reports linking autonomic remodeling with changes in DF and OI, even though these earlier studies did not record nerve activity directly and did not look at the temporal progression of AF.

Another key finding is the close relationship between atrial remodeling and atrial electrogram amplitude (voltage), with atrial voltage being closely correlated with AF progression even after 80 days of RAP. Since detailed tissue analysis indicates that both oxidative injury and fibrosis (fibrofatty remodeling) are progressive, and continue after 80 days of RAP, it is believed that progressive changes in atrial voltage at least partially reflect the underlying increase in inflammation-related changes in the fibrillating atrium. Indeed, increasing data implicates inflammation and related oxidative injury as a major molecular mechanism underlying the pathogenesis of AF. Excessive production of reactive oxygen species (ROS) is involved in electrical, structural, and/or autonomic remodeling. These remodeling processes lead to rapid focal firing and reentry and eventually to structural changes such as fibrosis with each process amplifying over time until AF becomes persistent. Although an increase in oxidative injury with AF is to be expected, the present system for the first time quantified the extent of oxidative injury over increasing duration of AF in the RAP model. A significant increase in DNA oxidative damage in the left atrial regions (LAA, PLA, and LAFW) was found with increasing duration of AF. The extent of fibrosis also increased with increasing AF (RAP days) in atrial sub-regions. Importantly, the extent of DNA oxidative damage appeared to be positively correlated to the presence of fibrosis.

Taken together, the current study suggests that AF electrograms—both frequency and amplitude measures—are able to accurately detect the onset and progression of the above-mentioned molecular mechanisms in real-time. As discussed herein, these findings have important translational implications for the clinical management of AF.

Imaging, such as T1/T2 MR imaging, is an important tool to study dynamic changes in the progression of electrical and structural remodeling in AF. A key motivation underlying the study was to understand how AF progression can be visualized in real-time. Although AF electrogram analysis is a dynamic analysis that can be used to study AF progression, AF electrograms typically can only be analyzed in detail at the time of electrophysiological study. It was therefore hypothesized that real-time AF progression can be studied non-invasively with MR-based techniques, with these techniques being synergistic to AF electrogram analysis and yielding complementary information on the AF disease state. The inventor elected to use novel MRI sequences, T1, T2, and ECV, and it is believed that these MRI parameters have not previously been monitored or considered for identifying changes in the left atrium.

It was found that T1 and T2 imaging demonstrates significant and progressive changes in AF as compared to normal atria. Furthermore, these changes in T1/T2 imaging appear to be linear over time, with changes continuing to be seen both in early (less than 80 days) and late-stage (after 80 days) AF remodeling, as shown in FIG. 6.

These temporal changes in T1 and T2 times with increasing duration of RAP correspond closely to the temporal increase in oxidative injury and interstitial fibrosis noted on histological analysis. Prior studies in the ventricle in models of cardiomyopathy have suggested that T2 times reflect tissue edema and inflammation, while ECV is more representative of interstitial fibrosis. The increase in T2 times with increasing duration of AF is therefore consistent with the progressive increase noted in oxidative injury in the fibrillating atrium. However, the lack of increase in ECV—thought to reflect interstitial fibrosis—is not consistent with the increase in interstitial fibrosis that was noted on histological analysis. While the reasons for this lack of change in ECV are not clear, one potential reason may be the need to register ROI's drawn on the native and post-contrast maps. Native T1 is measured on a single image, and the increase in T1 times-which reflects both increases in myocardial edema and fibrosis-supports the postulate that both interstitial edema/inflammation and interstitial atrial fibrosis contribute to the progressive changes noted on MRI. It is also possible that some aspects of atrial remodeling, such as alterations in ion channels and calcium cycling proteins in response to autonomic remodeling in the atria, may be reflected in the changes noted on T1/T2 MR times.

Despite the importance of autonomic nerve remodeling and inflammation/oxidative injury (and resulting structural changes such as fibrosis) in the genesis and maintenance of AF, it is currently not possible to detect autonomic nerve activity, inflammation, and oxidative stress-related changes in the fibrillating atrium in real-time. Not only does the proposed method shed light on the role of these molecular mechanisms in the progression of AF, but also provides new insights into how these molecular mechanisms can be potentially detected in the individual patient using a combination of AF electrogram techniques and novel MR tools such as T1/T2 imaging. A better accurate understanding of the AF pathophysiological state in the individual patient will allow for the development of novel therapeutic strategies for AF.

Thus, described herein is a system that combines AF electrogram characteristics and T1/T2 MRI to help accurately detect not only early-stage AF remodeling (secondary to autonomic remodeling and inflammation-related changes such as OS) but also more advanced AF remodeling due to increasing OS and fibrosis. The following abbreviations are used herein:
8-OHdG 8-hydroxy-2′-deoxyguanosine
AF Atrial fibrillation
ANOVA Analysis of variance
CMR Cardiovascular magnetic resonance imaging
CL Cycle length
DF Dominant Frequency
EF Ejection fraction
DNA Deoxyribonucleic acid
ECV Extracellular volume fraction
EGM Electrogram
LAA Left atrial appendage
LAFW Left atrial free wall
MOLLI Modified look-locker inversion recovery
MRI Magnetic resonance imaging
NaCl Sodium chloride
OCT Optimal cutting temperature compound
OI Organization Index
OS Oxidative stress
PLA Posterior left atrium
PNA Parasympathetic nerve activity
PRA Posterior right atrium
RAP Rapid atrial pacing
RAA Right atrial appendage
RAFW Right atrial free wall
ROS Reactive oxygen species
SD Standard deviation
SLGP Superior left ganglionic plexi
SSFP Steady-state free-precession
SEM Standard error of the mean
SV Stroke volume
V_{bip} Bipolar voltage

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A system to identify and predict atrial fibrillation, the system comprising:
a memory configured to store one or more electrograms and one or more nerve recordings of a patient; and
a processor operatively coupled to the memory and configured to:
identify one or more atrial fibrillation characteristics based on the one or more electrograms and the one or more nerve recordings, wherein the one or more atrial fibrillation characteristics include oxidative stress and fibrosis; and
identify a progression state of the atrial fibrillation based on the one or more atrial fibrillation characteristics.

2. The system of claim 1, wherein the one or more atrial fibrillation characteristics include autonomic remodeling.

3. The system of claim 1, wherein the memory is further configured to store one or more T1/T2 maps obtained through imaging, and wherein the one or more atrial fibrillation characteristics are identified based at least in part on the one or more T1/T2 maps.

4. The system of claim 1, wherein the processor predicts a subsequent progression state of the atrial fibrillation based on the identified progression state and the one or more atrial fibrillation characteristics.

5. The system of claim 1, wherein the memory stores an indication of whether the patient plans to pursue treatment of the atrial fibrillation, and wherein the processor predicts a survival rate for the patient over a period of time, wherein the survival rate is based on the indication of whether the patient plans to pursue treatment and the identified progression state.

6. The system of claim 1, wherein the processor is configured to identify an optimal treatment plan for the atrial fibrillation based on the identified progression state and the one or more atrial fibrillation characteristics.

7. The system of claim 1, wherein the processor predicts a time at which the atrial fibrillation will terminate and a normal sinus rhythm will commence for the patient, wherein the prediction is based on the identified progression state.

8. The system of claim 7, wherein processor also predicts a duration of the normal sinus rhythm.

9. The system of claim 1, wherein the one or more atrial fibrillation characteristics include cycle length, organization index, dominant frequency, and voltage.

10. The system of claim 1, wherein the processor uses telemetry to obtain and analyze the one or more nerve recordings.

11. The system of claim 1, wherein the one or more atrial fibrillation characteristics include oxidative stress levels in right and left atrial sub-regions of the patient, and wherein the progression state is identified based at least in part on the oxidate stress levels.

12. The system of claim 1, wherein the one or more atrial fibrillation characteristics include a fat percentage in heart tissue of the patient, and wherein the progression state is identified based at least in part on the fat percentage.

13. The system of claim 1, wherein one or more atrial fibrillation characteristics incudes nerve frequency, and wherein the processor quantifies nerve frequency based at least in part on a time between nerve peaks assessed with zero-crossings based on the one or more nerve recordings.

14. The system of claim 13, wherein the nerve frequency is based at least in part on an area under a nerve signal.

15. The system of claim 1, wherein the one or more electrograms include an intracardiac electrogram of the patient and a body surface electrogram of the patient.

16. A method for identifying and predicting atrial fibrillation, the method comprising:
storing, in a memory of a computing system, one or more electrograms and one or more nerve recordings of a patient;
identifying, by a processor of the computing system, one or more atrial fibrillation characteristics based on the one or more electrograms and the one or more nerve recordings, wherein the one or more atrial fibrillation characteristics include oxidative stress and fibrosis; and
identifying, by the processor, a progression state of the atrial fibrillation based on the one or more atrial fibrillation characteristics.

17. The method of claim 16, further comprising storing, in the memory, one or more T1/T2 maps obtained through imaging, wherein the one or more atrial fibrillation characteristics are identified based at least in part on the one or more T1/T2 maps.

18. The method of claim 16, further comprising predicting, by the processor, a subsequent progression state of the atrial fibrillation based on the identified progression state and the one or more atrial fibrillation characteristics.

19. The method of claim 16, further comprising:
storing, in the memory, an indication of whether the patient plans to pursue treatment of the atrial fibrillation; and
predicting, by the processor, a survival rate for the patient over a period of time, wherein the survival rate is based on the indication of whether the patient plans to pursue treatment and the identified progression state.

20. The method of claim 16, further comprising identifying, by the processor, an optimal treatment plan for the atrial fibrillation based on the identified progression state and the one or more atrial fibrillation characteristics.
